(19) United States
(12) Patent Application Publication
(10) Pub. No.: US 2005/0106741 A1
(43) Pub. Date: May 19, 2005

(54) Determination of aromatic hydrocarbons in edible oil products

(76) Inventor: Abraham Dijke, Poortugaal (NL)

Correspondence Address: PERKINS COIE LLP, P.O. BOX 2168, MENLO PARK, CA, 94026, US

(21) Appl. No.: 10/688,028

(22) Filed: Oct. 17, 2003

(51) Int. Cl. G01N 33/00

(52) U.S. Cl. 436/140

(57) ABSTRACT

Methods for rapid and precise analysis of low levels of aromatic hydrocarbons, particularly polycyclic aromatic hydrocarbons (PAH), such as benzo[a]pyrene (BaP), in edible oils, edible fats and related products, such as distillates of these materials, are described. The methods require minimal sample preparation and little or no operator input.

### FIELD OF THE INVENTION

The present invention relates to analysis of aromatic hydrocarbons, particularly polycyclic aromatic hydrocarbons (PAH), such as benzo[a]pyrene (BaP), in edible oils, edible fats and related products, such as distillates of these materials. In particular, it relates to a method and apparatus for such analysis employing in-line gel permeation chromatography (GPC)/high performance liquid chromatography (HPLC). Determination of sub-ppb levels of PAH, and determination of multiple PAH simultaneously, are described.

### REFERENCES

Cejpek, K. et al., Int. J. Environmental Anal. Chem. 61(1):65-80 (1995)
Germuska, R. and Michalski, R., Central European Journal of Public Health 8 (Suppl.):92-93 (2000)
Kraak, J. C. et al., Int. J. Environ. Anal. Chem. 25(1-3): 209-220 (1986)
Moret, S. and Conte, L. S., J High Resolut. Chromatogr. 21(4):253-257 (April 1998)
Moret, S. and Conte, L. S., J. Sep. Sci. 25(1-2): 96-100 (January 2002)
Moret, S. et al., J Chromatogr. A 750(1-2):361-368 (25 Oct. 1996)
Myojyo, K. et al., EP 632267 A1 (January 1995)
Perrin, J. L. et al., Fett Wiss. Technol. 95(2):40-51 (February 1993)
Sengupta, P. and Sil, S., J. Oil Technol. Assoc. India 15(3):54-55 (July-September 1983)
Vaessen, H. A. M. G. et al., Fresenius' Z. Anal. Chem. 332(4):325-332 (November 1988)
van Stijn, F. et al., J. Chromatogr. A 750(1-2): 263-273 (1996)
Williams, R. A. et al., J. Chromatogr. 477:315-315 (1989).

### BACKGROUND OF THE INVENTION

The presence of polycyclic aromatic hydrocarbons (PAH) in edible oils and fats, e.g. safflower oil, coconut oil, palm oil, etc., leads to high analysis cost in the processing of these materials. Source of such contamination include drying by direct heating with air that contains combustion gases. The use of direct-fired copra drying represents the main source of PAH present in crude coconut oil, which may include PAH levels exceeding 2,000 μg/kg. Refining of the oil reduces the content of the individual PAHs to a μg/kg level.

Detection and regulation of PAH in edible oils and fats is crucial, as many PAH are highly carcinogenic. FEDIOL (Féderation de l'Industrie d'Huilerie de la Communauté Europé enne) has adopted a limit of 25 ppb (25 μg/kg) for total PAH, 5 ppb for heavy PAH (five or more rings), and 1 ppb for BaP (benzo[a]pyrene) in edible fats and oils.

These oils may also contain monocyclic aromatic hydrocarbons (e.g. benzene, toluene, and xylenes) due to contamination with fuel oil, which also typically contains significant levels (about 10,000 ppm) of lower PAH (naphthalene, methyl naphthalene, phenanthrene, and acenaphthene). Conventionally, fuel oil contamination has been determined by GC/MS (gas chromatography/mass spectrometry) determination of aliphatic hydrocarbons. However, a certain percentage of these aliphatic hydrocarbons are already naturally present in edible oils. Accordingly, determination of fuel oil levels by analysis of aromatic hydrocarbons, which are not present in edible oil but represent an estimated 35% of most fuel oils, is expected to give more consistent results.

Many methods for the analysis of benzo[a]pyrene (B[a]p, or BaP) and other polycyclic aromatic hydrocarbons (PAH) in edible oil and fats have been reported. These generally employ one or more extraction, sample enrichment and/or clean-up steps, followed by quantitation using GC (gas chromatography) or HPLC. Extraction methods, generally employed to separate PAH from lipids, include solvent partition, caffeine complexation, and saponification, which is often followed by “clean up”, to remove other potentially interfering components, on a silica gel column (see review by Moret and Conte, 2000, and references cited therein). See also, for example, Sengupta et al. 1983; Moret et al. 1996, 1998, 2002; Perrin et al. 1993; Vaessen et al. 1988; Myojyo et al. 1995; Germuska et al. 2000; and Cejpek et al. 1995; all cited above. A protocol approved by FEDIOL for determination of BaP in edible oils is given in the Examples.

Most of these methods are time-consuming (up to 24 hours or more), expensive, and often incur loss of analyte and/or contamination of sample, leading to poor reproducibility. The amount of time required for analysis also results in the need for extensive storage of large amounts of material while awaiting the results of the analysis.

There is accordingly a need to provide more efficient means of analysis of such materials, in terms of time, expense, and material use, as well as methods that are reproducible and accurate at low levels of detection.

### SUMMARY OF THE INVENTION

The present invention provides, in one aspect, a method for determining the level of at least one polycyclic aromatic hydrocarbon (PAH) in a sample selected from edible oils, edible fats, and components thereof, e.g. fatty acids. The method comprises the steps of:
providing the sample in a first solvent in which the PAH is/are soluble;
applying the sample to a gel permeation chromatography (GPC) column, and eluting the sample with a GPC eluting solvent, effective to provide a fraction containing the PAH which is substantially free of triglyceride and free fatty acid components of the sample;
injecting the fraction, without isolation, into a GPC/HPLC interface, wherein a solvent in which the PAH have low solubility is added to the fraction;
transferring the fraction, without isolation, onto a reverse-phase high performance liquid chromatography (HPLC) column,
eluting the fraction, initially, with a solvent in which the PAH have low solubility,
separately eluting each PAH to be detected from the HPLC column with an HPLC eluting solvent,
detecting said at least one PAH, typically by fluorescence detection, at a wavelength(s) characteristic of said PAH, and
determining the level of said at least one PAH in the sample.

As used herein, a “solvent” may be a mixture of solvents, e.g. THF/acetonitrile or water/acetonitrile. The “solvent in which the PAH have low solubility” is preferably one in which they are less than 5% soluble. Such solvents include 95:5 to 100:0 water/acetonitrile.

Preferably, the method is carried out in an automated manner. In particular, the injecting and transferring steps are preferably initiated automatically by software control of a valve switching mechanism.

In one embodiment of the method, the eluting step comprises: pumping the GPC solvent through the GPC column, using a first pump, with output to a source at atmospheric pressure, which may be a refractive index (RI) detector, and, separately, pumping the HPLC solvent or solvent mixture, typically using a separate (second) pump, through an injection loop and thence through the HPLC column; the injecting step comprises: pumping GPC solvent containing the PAH, using the first pump, and the solvent in which the PAH have low solubility, typically using the second pump, via a mixing tee, to the injection loop, with output to RI detector or other source at atmospheric pressure, bypassing the HPLC column; and the transferring and eluting steps comprise: pumping the solvent in which the PAH have low solubility, followed by the HPLC solvent, typically using the second pump, through the injection loop and thence through the HPLC column.

The PAH may be selected from the group consisting of naphthalene, methyl naphthalene, fluorene, acenaphthene, acenaphthylene, phenanthrene, anthracene, fluoranthene, pyrene, benzo[a]anthracene, chrysene, benzo[a]fluoranthene, benzo[b]fluoranthene, benzo[k]fluoranthene, benzo[a]pyrene, benzo[ghi]perylene, dibenzo[ah]anthracene, indeno[123-cd]pyrene, benzo[e]pyrene, perylene, benzoperylene, anthanthrene, coronene, and combinations thereof. In selected embodiments, the PAH is selected from benzo[b]fluoranthene, benzo[k]fluoranthene, benzo[a]pyrene, benzo[ghi]perylene, dibenzo[ah]anthracene, indeno[123-cd]pyrene, benzo[e]pyrene, perylene, anthanthrene, coronene and combinations thereof. In one embodiment, a single PAH analyte, e.g. benzo[a]pyrene, is determined.

The first solvent mixture preferably comprises tetrahydrofuran (THF); an example is a mixture of THF and acetonitrile, preferably in a ratio of 70:30 or greater, e.g. about 70:30 to 75:25. The GPC eluting solvent is preferably THF. The “solvent in which the PAH have low solubility” is preferably 95:5 to 100:0 water/acetonitrile. The HPLC eluting solvent is typically a solvent gradient comprising varying ratios of water and acetonitrile.

Recovery of the PAH from the sample is typically at least 99%. The method can be used to detect a PAH, preferably benzo[a]pyrene, present in the sample at a level of less than 1 ppb, preferably at levels less than 0.5 ppb, and more preferably at levels less than 0.1 ppb (0.1 μg per kg).

In one embodiment, for detecting multiple PAH, detecting comprises switching a fluorescence detector to detection wavelengths characteristic of each PAH to be detected, at a predetermined HPLC retention time for the PAH, in an automated manner. The method can be used for determination of at least 10 different PAH in a sample, or, in other embodiments, for determination of at least 20 different PAH in a sample.

In a related aspect, the invention provides a rapid “fingerprinting” method for determining the level of benzo[a]pyrene in a sample selected from edible oils, edible fats and components thereof, the method comprising:
providing the sample in a solvent mixture comprising THF or acetonitrile;
applying the sample to a gel permeation chromatography (GPC) column;
eluting the sample with THF, effective to provide an aromatic hydrocarbon-containing fraction which is substantially free of triglyceride and free fatty acid components of the sample; and
measuring the fluorescence absorption of the fraction at wavelength(s) characteristic of benzo[a]pyrene, e.g. 378 nm excitation/403 nm emission. The method can be used to detect benzo[a]pyrene in a sample at levels less than 10 ppb, and preferably less than 5 ppb. The recovery of benzo[a]pyrene from said sample is preferably at least 99%.

The invention also provides a method for determining the level of aromatic hydrocarbons in such a sample, the method comprising:
providing the sample in a first solvent comprising THF or acetonitrile;
applying the sample to a gel permeation chromatography (GPC) column;
eluting the sample with THF, effective to provide an aromatic hydrocarbon-containing fraction which is substantially free of triglyceride and free fatty acid components of the sample; and detecting the level of the aromatic hydrocarbons in the fraction by UV absorption. Typically, the method further comprises the step of estimating the level of fuel oil contamination in the sample, based on a separately determined correlation between fuel oil level and UV absorption intensity of the fraction.

When the first solvent is acetonitrile, the method further comprises extracting the sample with the acetonitrile, and the extract of the sample is applied to the GPC column. The elution solvent is typically THF. The aromatic hydrocarbon fraction may contain one or more components selected from benzene, toluene, ethylbenzene, xylenes, naphthalene, methyl naphthalenes, phenanthrene, and acenaphthene.

These and other objects and features of the invention will become more fully apparent when the following detailed description of the invention is read in conjunction with the accompanying drawings.

### BRIEF DESCRIPTION OF THE DRAWINGS

FIG. 1 shows the output of GPC (gel permeation chromatography) analysis of a crude coconut oil sample having a BaP level of 55 μg/kg, with benzo[a]pyrene (and total PAH) eluting at about 9.7 minutes;

FIG. 2 shows a calibration curve for BaP in coconut oil matrix, prepared from seven standards ranging in concentration from 12 to 123 μg/kg (ppb);

FIG. 3 shows GPC chromatograms (superimposed) used for benzo[a]pyrene analysis of three samples of coconut oil;

FIG. 4 shows an overlay of GPC chromatograms of seven fuel oil standards, prepared by spiking palm oil with pure diesel oil, in concentrations ranging from 2 to 85 μg/g (ppm);

FIG. 5 is a linear calibration curve of ppm fuel oil vs. absorption at 226 nm, prepared using the data of FIG. 4;

FIGS. 6A-B are schematic diagrams of a solvent switching interface, in operation mode and in injection mode, respectively;

FIG. 6C is a schematic diagram of the in-line GPC/HPLC system, with the solvent switching interface in operation mode (per FIG. 6A);

FIG. 7 is a graph comparing benzo[a]pyrene levels measured in several samples, as determined by two commercial laboratories, and in accordance with an embodiment of the present invention;

FIG. 8 is an HPLC chromatogram showing separation of various PAH in a crude coconut oil sample, as obtained by in-line GPC/HPLC;

FIG. 9 is an HPLC chromatogram of a standard containing 34 ppm fuel oil in crude palm oil, as analyzed by in-line GPC/HPLC employing a single detection frequency set for detection of BaP; and

FIG. 10 is an HPLC chromatogram showing separation of multiple PAH in an edible oil sample, as obtained by in-line GPC/HPLC employing programmed switching of detection frequency for each component PAH.

### DETAILED DESCRIPTION OF THE INVENTION

The present invention provides methods for the detection of low levels of aromatic hydrocarbons, particularly benzo[a]pyrene and/or other PAH, in crude and refined edible oils or fats, as well as related products, such as fatty acids derived from such oils or fats. The methods employ gel permeation chromatography (GPC) alone or in combination with reverse phase high performance liquid chromatography (RP-HPLC).

I. Analytes

Polycyclic aromatic hydrocarbons (PAH) are organic compounds containing two or more fused carbocyclic aromatic rings. These compounds arise from geochemical processes and are also frequent byproducts of incomplete combustion or pyrolysis of organic matter. Many are known to be carcinogenic. They are classified as light PAH, having two to four, generally three or four, aromatic rings, or heavy PAH, having five or more fused rings. The former class includes, for example, naphthalene, fluorene, acenaphthene, acenaphthylene, phenanthrene, anthracene, fluoranthene, pyrene, benzo[a]anthracene, and chrysene. The latter class includes, for example, benzo[a]fluoranthene, benzo[b]fluoranthene, benzo[k]fluoranthene, benzo[a]pyrene, benzo[ghi]perylene, dibenzo[ah]anthracene, indeno[123-cd]pyrene, benzo[e]pyrene, perylene, benzoperylene, anthanthrene, and coronene. Benzo[a]pyrene is reported to be the most carcinogenic of known PAH.

Of these compounds, the following appear on the FEDIOL list:
Phenanthrene
Anthracene
Fluoranthene
Pyrene
Benzo[a]anthracene
Chrysene
Benzo[e]pyrene
Perylene
Benzo[a]pyrene
Dibenzo[ah]anthracene
Benzoperylene
Anthanthrene
Coronene.

Any other PAH desired to be detected may also be detected by the methods described herein, once a retention time and detection wavelength(s) are known or determined for the particular compound. The method may also be used to detect alkyl substituted PAH, such as methyl naphthalene.

Fuel oils are complex mixtures of alkanes, alkenes, cycloalkanes, and aromatic hydrocarbons, and also include a low percentage of sulfur-, nitrogen- and oxygen-containing compounds. Aromatic compounds represent an estimated 35% of fuel oils and generally include benzene, toluene, ethylbenzene and xylenes, known collectively as BTEX. Fuel oils also contain about 15000 ppm (1.5%) PAH, of which naphthalene, methyl naphthalenes, phenanthrene and acenaphthene make up about 10000 ppm (1.0%). Since BTEX, PAH and alkylated PAH, all present in fuel oils, are recognized hazardous components, toxicity assessment and confirmation of fuel oil contamination would optimally be based on the analysis of these aromatic compounds, rather than on analysis of saturated hydrocarbons, as in current practice. In addition, as noted above, these aromatic components do not occur naturally in edible oils, in contrast to saturated hydrocarbons.

II. Matrices

The materials analyzed in the methods disclosed herein are typically edible fats or oils, which may be of plant origin, including, for example, corn oil, coconut oil, sunflower oil, palm oil, safflower oil, and cacao products, or of animal origin, including, for example, lard, tallow, and fish oil. These materials are largely made up of various mixed triglycerides (TG); i.e. fatty alkyl triesters of glycerol, where the fatty alkyl components include such groups as palmityl, stearyl, myristyl, etc., and generally also include a free fatty acid (FFA) fraction. Edible oils, edible fats, and their components, such as FFA, may be referred to collectively herein as “edible oil products”.

In crude coconut oil, which is a representative edible oil, the average molecular weight of the triglyceride component is 674, and that of the free fatty acid (FFA) component, which is primarily C12 fatty acids, is 200. The average molecular weight of the triglyceride components of other representative edible oils is as follows: palm oil, 850; soybean oil, 875; sunflower oil, 879; rapeseed oil, 880; cottonseed oil, 860; and corn oil, 887.

The methods disclosed herein are useful for any matrix which is sufficiently soluble in the solvent(s) employed for isolation of an aromatic hydrocarbon component from the remaining matrix components by GPC, as described below, and whose components are sufficiently separable from the aromatic component via the disclosed GPC method.

Matrices having less than complete solubility in a given solvent may also be used, as described in Section IIIC below, where the analyte is extracted from the matrix. Such Typical matrices may include, for example, components of edible oils, such as fatty acids, and other food products, which may be pretreated to remove proteins or any other interfering components. Other hydrocarbon containing matrices, such as non-food oils, fuels, and lubricants, are also considered.

III. Analytical Methods: GPC

A. Isolation of Aromatic Hydrocarbons from Matrices by GPC

Gel permeation chromatography (GPC) is a form of liquid chromatography in which molecules are separated by repeated exchange between the solvent of the mobile phase and the solvent in the stagnant liquid phase within the pores of the column packing. The pore-size range of the packing material determines the molecular-size range within which separation can occur. In general, large molecules are excluded from some or all of the porous matrices of the packing by virtue of their physical size, and elute from the column before smaller molecules, which have the opportunity to permeate into a greater percentage of the pores of the solvent-filled matrix. The GPC separation also depends on actual three dimensional size or shape of the molecule; accordingly, molecules having similar molecular weights but different shapes can often be separated, and molecules having different molecular weights but similar shapes may elute together.

In the case of edible oils, a complete separation between triglycerides, FFA and aromatic hydrocarbons can be achieved by GPC as described herein. Because the aromatic compounds are similar in shape, they generally elute together despite differences in molecular weight.

GPC columns are commercially available in a variety of molecular weight capacities. For the present applications, separation is carried out on an analytical column having a relatively low effective molecular weight range, e.g. 0-1000. A preferred column packing material is crosslinked polystyrene, or styrene-divinylbenzene copolymer (SDVB). One such column is a Waters Styragel®-HR 0.5 column (4.3 mm I.D.×300 mm; 5μ particle size). Other suitable columns are available from various sources, e.g. Polymer Laboratories Inc. or Jasco Corporation.

Operation at a slightly elevated constant temperature was found to improve reproducibility of GPC results, and also results in better solubility of samples and lower column pressure. Preferably, a column temperature of about 40° C. is maintained during separation by the use of a column oven.

B. Rapid “Fingerprint” Analysis by GPC

In accordance with one embodiment of the invention, a “fingerprint” analysis of a single PAH, preferably benzo[a]pyrene, in a crude oil can be carried out in about 15 minutes or less, with a detection limit of about 5 μg/kg. In a typical analysis, the oil is dissolved in an appropriate solvent, e.g. a mixture of THF/acetonitrile, filtered through a 0.20 μm syringe-filter, injected onto the GPC column system, and eluted, preferably with THF.

Solvents or solvent mixtures are used, for dissolution of sample and for elution, in which all analytes are freely soluble. An exemplary solvent is THF. Use of a mixture of THF:acetonitrile in a ratio of about 5:2 by volume for sample preparation, and THF for elution, was found to increase the resolution and sharpness of the PAH peak, relative to sample preparation in THF alone. The ratio is preferably between about 70:30 and 75:25. Higher amounts of THF may be used, up to 100% THF, although peak sharpness may be diminished. Higher amounts of acetonitrile are not recommended due to diminished solubility of the analytes.

Fluorescence detection is preferred for detection of PAH, since the compounds tend to be strongly fluorescent, unlike the matrix components. Separate refractive index detection can be used to determine total sample injected, since the output signal of the RI detector depends only on the amount of sample. Therefore, the use of an internal standard is not necessary. Preferably, matrix components are also freely soluble in the sample and elution solvents.

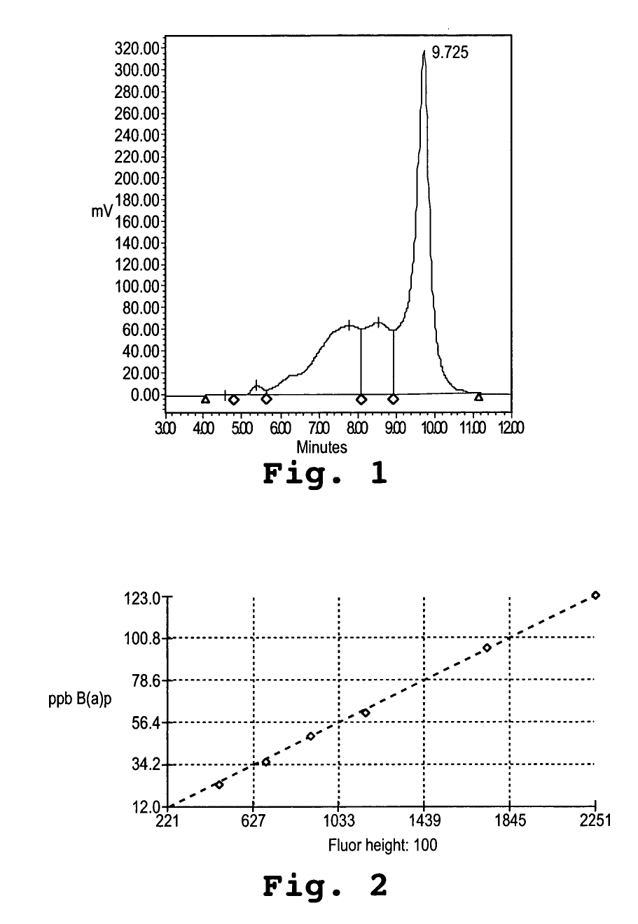

As noted above, PAH generally elute together in GPC processing of edible oils or similar matrices. Because each PAH has distinct excitation and emission wavelength maxima, fluorescence detection can be set for a specific compound, such as BaP, as shown in FIG. 1.

About eight other PAH compounds were found to give some level of interference with BaP at this setting, as described in Example 1 below. Anthracene is the only compound of those shown giving a potentially significant amount of interference at these wavelengths.

FIG. 1 shows the output of GPC analysis of a crude coconut oil sample having a BaP level of 55 μg/kg, with BaP (and total PAH) eluting at about 9.7 minutes. The primary components of the edible oil, TG and FFA, are not detected by a fluorescence detector, as noted above. However, they can be seen to elute at about 6.7 and 7.4 minutes, respectively, using a refractive index detector.

To prepare a calibration curve for a coconut oil matrix, seven BaP standards were prepared ranging in concentration from 12 to 123 μg/kg. The calibration curve (prepared using Cargill Instrument Performance Monitoring software) is shown in FIG. 2.

Three samples of crude coconut oil were analyzed for BaP using the present GPC method. Twenty duplicate runs were made, and the results were compared with analyses by independent laboratories. The results, given in Table 1, show a low standard deviation for the GPC method and reasonable agreement with the external analyses.

TABLE 1

| | Sample No. | | |
|---|---|---|---|
| | 1 | 2 | 3 |
| B[a]P conc. det'd by external lab | 9.5 μg/kg | 21.7 μg/kg | 73.0 μg/kg |
| B[a]P conc. by GPC, mean value | 9.3 μg/kg | 20.4 μg/kg | 70.8 μg/kg |
| Standard deviation | 0.4 | 0.9 | 1.1 |

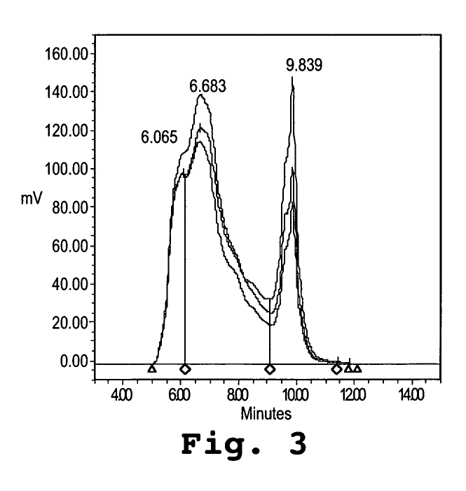

If desired, a separate BaP calibration curve may be prepared for different matrices in practicing the present GPC analysis, since different matrices may contain the various PAH in different proportions. However, a calibration curve prepared for a coconut oil matrix was shown to give accurate results in analysis of crude sunflower oil. Analysis of three samples of sunflower oil for BaP at levels below about 10 μg/kg, using the calibration curve prepared with coconut oil, showed good correlation with outside laboratory figures. FIG. 3 shows the chromatograms for these three samples superimposed, where the samples were determined by an external laboratory to contain, respectively, 5.3, 6.3, and 8.9 μg/kg BaP. Determination using the present method, using a calibration curve prepared with a coconut oil matrix, gave levels of 5.7, 6.4, and 8.1 μg/kg, respectively.

This method of rapid analysis is found to be highly reproducible, although determination of BaP may depend on the presence of anthracene. The detection limit for BaP using this method is about 5 ppb (μg/kg).

C. GPC Analysis of Fuel Oil Contamination by Detection of Lower Aromatic Hydrocarbons

For analysis of lower aromatic hydrocarbons, such as BTEX, UV detection is used, in the 200-750 nm wavelength range. The compounds elute close together in GPC analysis, and are detectable at about 226 nm wavelength.

To enhance detection of these compounds in a typical matrix, such as an edible oil, it may be desirable to increase their concentration by extraction of the matrix with acetonitrile. Triglycerides are poorly soluble in acetonitrile, while the different aromatic compounds in fuel oil (and free fatty acids) are soluble. Extraction is carried out by agitating a mixture of the crude oil and acetonitrile (e.g. one gram oil per 6 ml solvent) for several seconds and allowing the mixture to settle. The acetonitrile (upper) phase is separated, filtered (e.g. through a 0.45 μm syringe filter) into a sampler vial, and used for injection into the GPC system. This treatment typically increases the concentration of the analytes in an edible oil matrix by a factor of about 25. THF is preferred for elution of the sample.

Pure diesel oil (B.C.R. Standard No. 106, Community Bureau of Reference, Bruxelles) was used to prepare seven standards of fuel oil in crude palm oil, ranging in concentration from 2 to 85 μg/g. This concentration range brackets the concentration of 25 ppm fuel oil determined in an agreement between refiners to be the maximum level of fuel oil contamination acceptable for edible oil production. The standards were analyzed after the treatment described above (agitation with acetonitrile) used for sample preparation.

In the crude palm oil used for preparation of standards, 6 μg/g (6 ppm) aliphatic hydrocarbons was found by an external commercial lab, according to the AOCS method. Because palm oil generally contains about 4 to 20 ppm aliphatic hydrocarbons, the oil was considered free of fuel oil contamination, based on this determination.

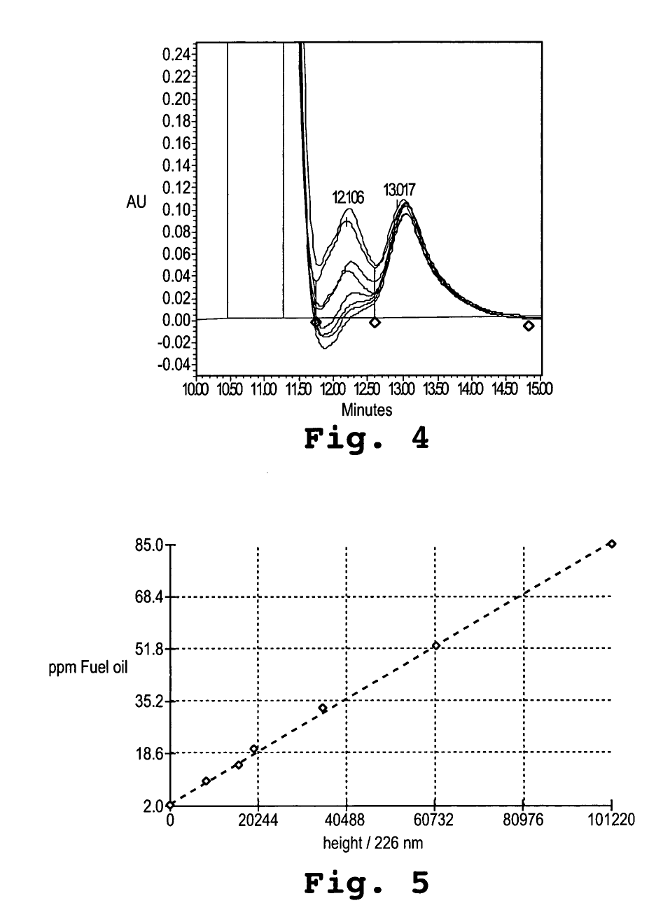

FIG. 4 shows the overlay of the GPC chromatograms of the seven standards, with UV detection at 226 nm. Under the conditions of analysis, the aromatic compounds eluted at about 12.2 minutes.

A linear calibration curve of ppm fuel oil vs. absorption at 226 nm (FIG. 5) was prepared from these standards, using the Cargill Instrument Performance Monitoring software. Such a calibration curve gives the correlation between the level of aromatic hydrocarbons as determined by UV absorption and ppm fuel oil, for fuel oil of similar composition to that used in prepared the standards.

The reproducibility of the GPC method for fuel oil contamination was tested using six replicates of three different standards. The results are given in Table 2. Detection limit for this method was found to be about 2 ppm.

TABLE 2

| | Conc. diesel oil in standard, ppm | | |
|---|---|---|---|
| | 15 | 53 | 85 |
| Mean value (μg/g) of six analyses | 15.7 ± 0.5 | 53.9 ± 0.6 | 84.5 ± 0.9 |

IV. Analytical Methods: In-line GPC/HPLC

A. Background, Apparatus, and General Method

Separation of compounds by HPLC, as well as other modes of liquid chromatography, is based on interaction between the compounds, dissolved in a mobile phase, and the stationary phase, i.e. the solid column packing. The driving force of the separation is the difference in enthalpy of interactions of the different species (compounds, mobile phase, and stationary phase). Reversed phase (RP) chromatography, which employs a nonpolar (hydrophobic) stationary phase and a polar mobile phase, is preferred for the present separations. In RP-HPLC, the primary interactions responsible for retention and separation are noncovalent associations of non-polar moieties (the analytes, or nonpolar components thereof, and the stationary phase) in polar media. The typical mobile phases used in RP-HPLC are aqueous solutions of displacement agents, such as acetonitrile or 1-propanol. Typically, analytes are eluted with a mobile phase gradient in which the concentration of the displacement agent increases gradually.

The most widely used RP-HPLC adsorbents in the field are based on hydrocarbon modified silica, typically silica modified with octadecyl (C18) groups. Such columns may be referred to as ODS (octadecyl silica) columns, and many such columns are commercially available; e.g. Waters Bondapack C18. Columns containing alkylphenyl bonded silica have also been described as useful for separation of chemical compounds including PAH (e.g., Faizy et al., U.S. Pat. No. 5,993,653).

For elution, a solvent gradient consisting of polar solvents is used. An exemplary gradient contains acetonitrile and water, though other polar elution solvents, e.g. methanol or ethyl acetate, may also be used. In view of the strong fluorescence exhibited by most of the PAH analytes, a spectrofluorometric detector is preferred.

The PAH fraction obtained via GPC separation from matrix components, such as TG and FFA in an edible oil matrix, as described above, is free of any detectable triglyceride or FFA component. This fraction is transferred in-line, that is, without isolation of the fraction or removal from the apparatus, to the HPLC system via a GPC-HPLC interface.

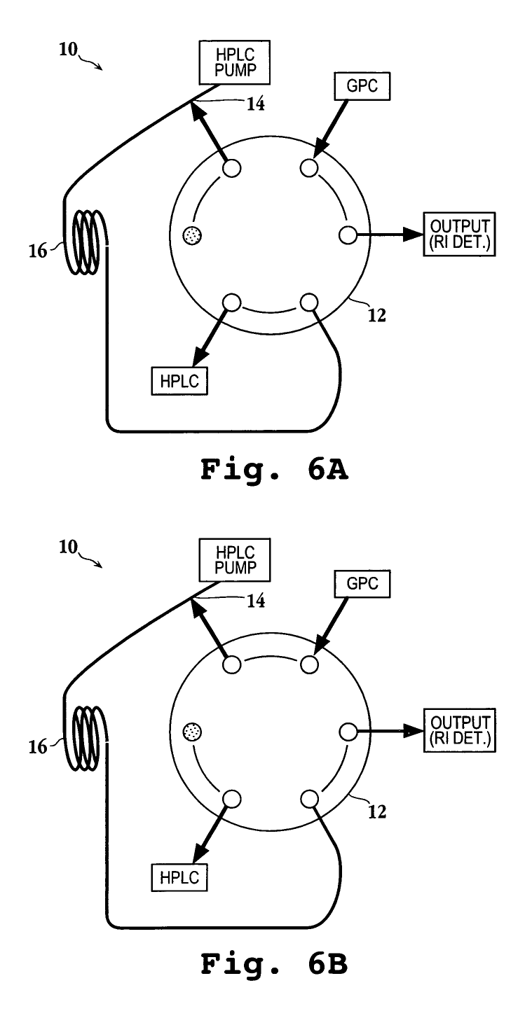

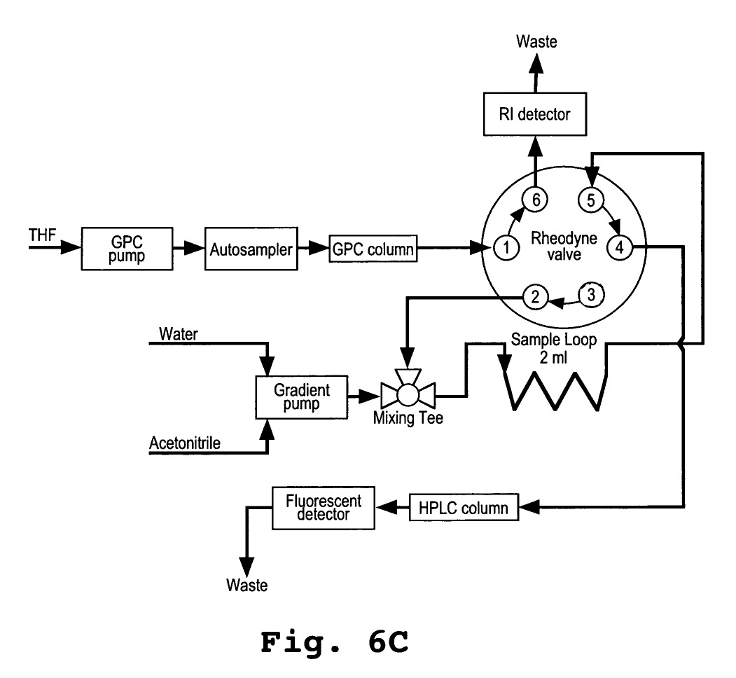

An exemplary interface, such as illustrated at 10 in FIGS. 6A-B, includes an injection switching mechanism 12, such as a Rheodyne automated six-position fluid processor, a mixing tee 14, and an injection loop 16, preferably having a volume of 2 mL. A schematic illustration of the components of the in-line GPC-HPLC system, with the interface in separation mode (as described below), is shown in FIG. 6C.

One function served by the interface is the switching of solvents between the GPC and HPLC systems. As described above, solvents in which the PAH are highly soluble, such as THF, are preferred for the GPC separation from other matrix components. In a typical GPC separation of trace amounts of PAH in an edible oil sample, the PAH fraction is obtained in about 0.3 mL of THF. Direct injection of this fraction into the HPLC column would generally result in rapid elution of PAH essentially as a single peak. For separation on the reverse-phase HPLC column, a polar solvent gradient, as noted above, is employed.

Coupled GPC-HPLC systems have been described in the literature. Some employ similar solvents in the two systems (e.g. Winkle 1990; De Vries 1989), so that solvent switching is not required. In cases where different solvents are employed, contacting of the GPC solvent with the HPLC column is described as problematic. See e.g. Kraak et al. 1986, or van Stijn et al. 1996, which notes a similar problem in a DACC (donor-acceptor complex chromatography)—HPLC coupled system. Kraak et al. addressed the problem, in a system for removal of proteins from plasma samples, by incorporating a precolumn to absorb analytes from the GPC solvent and then release them into the HPLC mobile phase. Williams et al. (1989) describe a method in which a GPC eluate in THF is continuously diluted with water, and a selected fraction is transferred to an RP-HPLC column in water. This system was described as having a detection limits of about 50 ppb.

In the present system, as noted above, GPC is carried out in an organic solvent such as THF, while an aqueous solvent system is generally employed for HPLC separation. Accordingly, an aqueous solvent is added to the sample within the interface, which comprises a valve switching device. This solvent is one in which the PAH to be detected have low solubility (also referred to as a “non-solvent”), e.g. 95:5 to 100:0 water/acetonitrile. In injection mode, as shown in FIG. 6B, the PAH fraction in THF (typically having a volume of about 0.3 ml, as noted above) is mixed with such a solvent, at substantially the same volume and flow rate, via mixing tee 14 and transferred to injection loop 16.

Another function of the interface is protection of the GPC column from rapid changes in pressure when the sample is injected onto the HPLC-system. During injection, as shown in FIG. 6B, both the GPC THF stream and the HPLC water/acetonitrile stream are output to the RI detector, which operates at atmospheric pressure. Accordingly, the pressure on the GPC column remains substantially constant.

Following injection (about 1 minute), the valve settings return to normal operation (FIG. 6A, and the contents of injection loop 16 are transferred onto the HPLC column. Due to the presence of added water in the sample, the PAH analytes are no longer soluble and deposit at the top of the HPLC column. The PAH are initially eluted using a “non-solvent” as described above, followed by a programmed solvent gradient, effective to separately elute each PAH to be detected from the HPLC column.

Accordingly, the sequence of operations in a typical in-line GPC-HPLC interface switch, which is preferably carried out in an automated manner, is as follows.

(1) GPC phase (FIG. 6A): GPC solvent is pumped through the GPC column (GPC pump, not shown), with output to the RI detector or other source at atmospheric pressure; separately, HPLC gradient or “non-solvent” is pumped (HPLC pump) through injection loop 16 and the HPLC column.

(2) Injection phase (FIG. 6B): GPC solvent containing analytes (GPC pump) and “non-solvent” (HPLC pump) are pumped via mixing tee 14 to injection loop 16, with output to the RI detector or other source at atmospheric pressure; the HPLC column is bypassed. Injection is preferably carried out for a programmed or predetermined period of time, e.g. one minute.

(3) Separation phase (FIG. 6A): “Non-solvent”, followed by HPLC gradient, is pumped through injection loop 16 (HPLC pump), transferring the contents onto the HPLC column for elution.

Preferably, switching from phase (1) to (2), and from (2) to (3), is done automatically (i.e. without the need for operator input) via software control of the injection switching mechanism 12. The device is thus programmed to switch to injection mode (FIG. 6B) after a predetermined time following injection; this predetermined time is based on the predetermined GPC retention time(s) of the analyte(s). The device is then automatically switched to separation mode (FIG. 6A) after a predetermined injection time, e.g. one minute.

B. Quantitative Analysis of Benzo[a]pyrene by GPC-HPLC

In one aspect, the invention provides a method for accurate determination of a single PAH, such as benzo[a]pyrene, in complex matrices such as edible oils and fats. This method employs tandem, in-line GPC-HPLC to isolate and/or quantify the analyte. A GPC separation step, such as that described above for fast “fingerprint” analysis of BaP, is used as a preliminary purification step in this method. The use of HPLC allows detection of “heavy” PAH, which typically cannot be detected by gas chromatography (GC) methods due to thermal decomposition. The method has a detection limit of about 0.01 μg/kg, and can be carried out in about 45 minutes or less.

As described further below, the invention also provides a method for accurate determination of multiple PAH in a single analysis. The determination of 20 different PAH, carried out in about 100 minutes or less, is described in Section D below.

In an illustration of the GPC-HPLC method for quantitation of BaP, standards containing BaP at levels ranging from 1 to 80 ppb in refined coconut oil were prepared. The BaP was obtained from Alltech Chemie. The conditions described in Example 3, below, including a gradient program found particularly effective for isolation of benzo[a]pyrene, were employed for HPLC separation.

A calibration curve was prepared from the different standards, followed by ten duplicate runs of the 30 ppb standard. The mean value of BaP concentration obtained from the ten duplicate runs of the 30 ppb standard was 29.61 ppb, with a standard deviation of 0.58 ppb.

In a further illustration of the method, BaP levels in several field samples were analyzed under similar conditions, and the results were compared with analyses by two independent laboratories. These laboratories are expected to have employed the conventional protocol approved for determining BaP levels in edible oils, which is given in the Examples. As noted above, this conventional protocol is much less efficient in terms of time, labor and materials than the presently disclosed method.

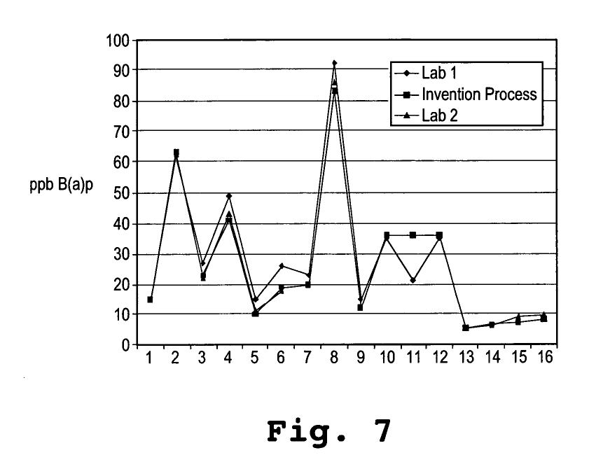

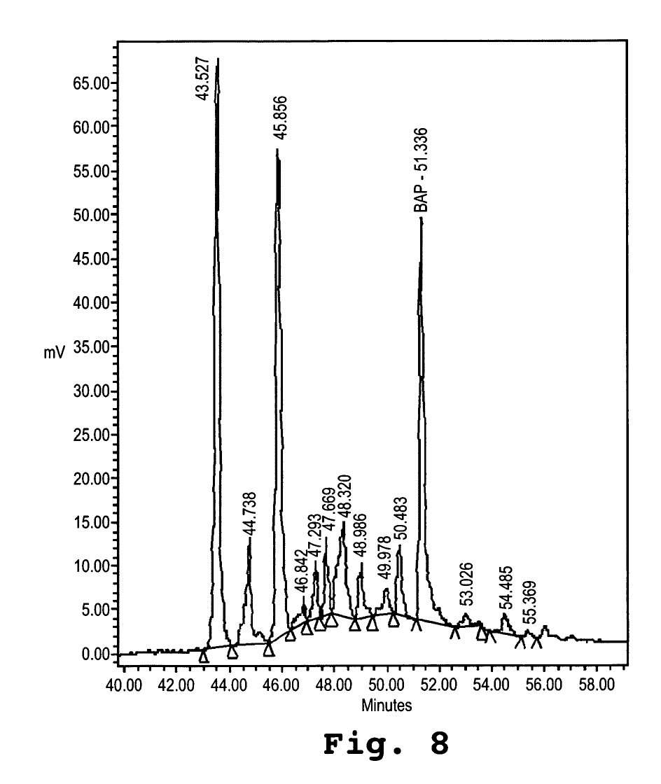

As shown by Table 3 below and graphically in FIG. 7, the method of the invention gave good agreement with commercial testing. Sample nos. 1-12 were coconut oil samples, while samples 13-15 were sunflower oils, showing that the disclosed method can be used with various edible oil matrices.

The Table also shows values obtained from GPC alone, which are often in good agreement with the more quantitative method.

TABLE 3

| | | Comm. | Comm. | Inline GPC- | GPC |
|---|---|---|---|---|---|
| Sample | Sample | Lab 1 | Lab 2 | HPLC | alone |
| No. | Description | BAP level, ppb | | | |
| 1 | Coconut oil | 15 | | 15 | |
| 2 | Coconut oil | 62 | | 63 | |
| 3 | Coconut oil | 27 | 22 | 23 | 23 |
| 4 | Coconut oil | 49 | 43 | 41 | 42 |
| 5 | Coconut oil | 15 | 11 | 10 | 13 |
| 6 | Coconut oil | 26 | 18 | 19 | 21 |
| 7 | Coconut oil | 23 | | 20 | |
| 8 | Coconut oil | 92 | 86 | 83 | 79 |
| 9 | Coconut oil | 15 | | 12 | |
| 10 | Coconut oil | 35 | | 36 | |
| 11 | Coconut oil | 21 | | 36 | |
| 12 | Coconut oil | 35 | | 36 | |
| 13 | Sunflower oil | | 5.3 | 5.7 | 5.7 |
| 14 | Sunflower oil | | 6.3 | 6.6 | 6.4 |
| 15 | Sunflower oil | | 8.9 | 8.1 | 8.1 |

Table 4 shows further comparative data, this time for analysis of refined coconut oil samples having BaP levels less than 3.0 ppb.

TABLE 4

| | | | In-line |
|---|---|---|---|
| | Comm. | Comm. | GPC- |
| Sample | Lab 1 | Lab 2 | HPLC |
| No. | BaP level in ppb | | |
| 16 | — | 2.50 | 2.50 |
| 17 | — | 1.20 | 1.10 |
| 18 | 0.50 | — | 0.58 |
| 19 | 0.00 | — | 0.24 |
| 20 | 0.00 | — | 0.55 |
| 21 | 0.00 | — | 0.18 |
| 22 | 0.20 | — | 0.34 |
| 23 | 0.00 | — | 0.22 |

FIG. 8 shows separation of various PAH from a crude coconut oil sample, using GPC/HPLC as described, with fluorescence detection at 378 nm/403 nm, as described in Example 3. The HPLC solvent gradient shown in Example 3 was optimized for separation of BaP from other components. Good separation was obtained, with BaP eluting at 51.3 minutes in this example. Total analysis time, including sample preparation, was about 80 minutes. The solvent gradient can be varied to optimize isolation of other single PAH compounds as desired.

C. GPC-HPLC Analysis of Multiple PAH: Single Detection Setting

Aromatic compounds isolated from edible oil matrices by GPC for detection of fuel oil contamination, as described in Section IIIC above, can be analyzed further by in-line HPLC. In an illustration of this method, an aromatic hydrocarbon-containing GPC fraction, obtained as described in Section IIIC, was analyzed for the compounds methyl naphtalene, acenaphthene and phenanthrene, which are the predominant PAH in fuel oil. The interface and HPLC apparatus were as described above and in Materials and Methods. The following gradient elution program was used:

| Time, min | Flow, ml/min | Acetonitrile (%) | 5:95 ACN/Water (%) |
|---|---|---|---|
| 0.00 | 0.50 | 0 | 100 |
| 9.50 | 0.50 | 0 | 100 |
| 11.40 | 0.30 | 0 | 100 |
| 13.10 | 0.30 | 0 | 100 |
| 14.00 | 0.50 | 0 | 100 |
| 55.00 | 0.50 | 100 | 0 |
| 75.00 | 0.50 | 100 | 0 |
| 75.50 | 0.50 | 0 | 100 |
| 90.00 | 0.50 | 0 | 100 |

The analytes were detected by fluorescence at 244/375 nm (excitation/emission).

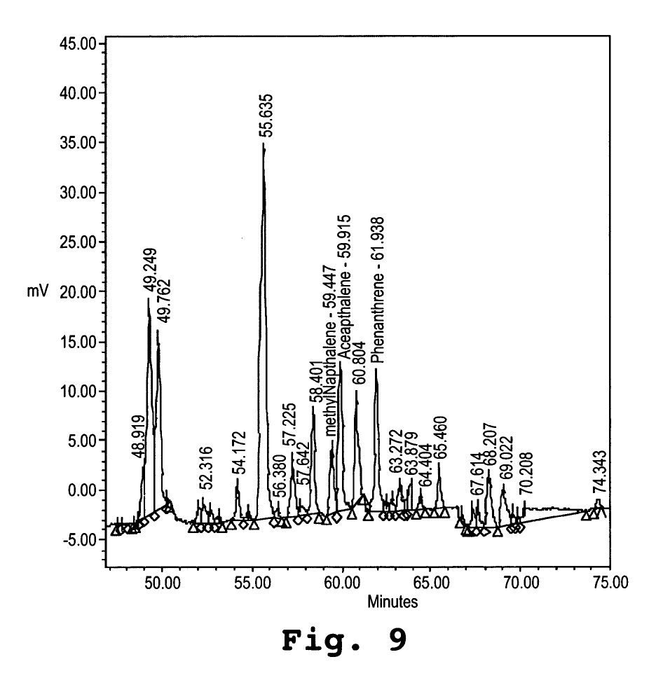

The seven standards prepared as described in Section IIIC, having 2 to 85 ppm fuel oil in crude palm oil, were analyzed by GPC/HPLC. FIG. 9 shows fluorescence detection output of the fraction containing 34 ppm fuel oil. As shown, the HPLC retention times of methyl naphthalene, acenaphthene and phenanthrene were 59.5 min, 60.0 and 62.0 minutes, respectively.

A calibration curve for ppm fuel oil vs. level of these three PAH was prepared, and the curve was used to estimate ppm fuel oil in several commercial samples by analyzing these three PAH by in-line GPC/HPLC. The results, including results obtained by GPC only, are shown in Table 5.

As shown, at these low levels, the commercial results, obtained by analyzing for aliphatic hydrocarbons, tend to be significantly higher than those obtained by analysis of aromatics. As noted above, this is likely due to the presence of naturally occurring aliphatic hydrocarbons in edible oils.

TABLE 5

| Sample No. | | GPC/HPLC Analysis | Commercial |
|---|---|---|---|
| (Palm Oil) | GPC Analysis | (Three PAH) | Analysis |
| 24 | 1 ppm | 1.5 ppm | 12 ppm |
| 25 | 0 | 1.0 | 6 |
| 26 | 2 | 3.0 | 9 |
| 27 | 0 | 0 | 6 |
| 28 | 0 | 0.2 | 7 |

D. GPC-HPLC Analysis of Multiple PAH: Multiple Detection Settings

For optimal detection of multiple PAH, the fluorescence detector may be switched to specific wavelengths as appropriate. This method allows rapid determination of levels of multiple PAH in a sample in a single analysis.

Calibration standards, prepared by spiking blank oil with known amounts of each PAH to be determined, can be used to determine retention time and detector response for each PAH for a given column setup and conditions. The sample to be analyzed is accurately weighed and prepared as described above, e.g. by dissolving in 5:2 THF/acetonitrile and filtering, and injected onto the GPC column. The sample is eluted with THF, then injected into the sample loop as described above (see FIG. 6B), where it is mixed with a solvent in which the PAH have low solubility (“non-solvent”), e.g. 95:5 water/acetonitrile. The sample is then transferred to the HPLC column (by switching back to operation mode; FIG. 6A), and the HPLC gradient is initiated. The elution typically begins with the “non-solvent”, as shown in Example 4 below.

The fluorescence detector is programmed to switch to appropriate detection wavelengths for each component (PAH) at the predetermined HPLC retention time of that component. One such program is shown in Example 4 below. (Because acenaphthylene is not detectable by fluorescence, it may be detected by incorporating a UV detector into the system.) Compounds are identified by comparing retention time with the previously injected standard and are quantified by peak area measurement.

As an illustration, a sample containing twenty PAHs which have been found in edible oils, including the thirteen PAHs on the FEDIOL list, was analyzed in about 100 minutes, as described in Example 4 below.

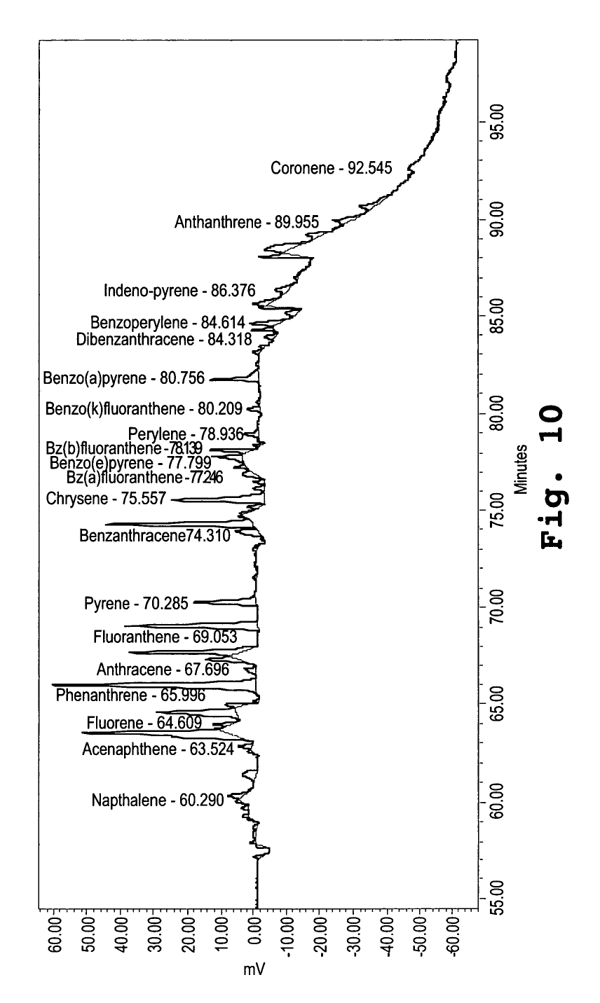

A chromatogram of a typical multi-PAH analysis, using multiple detector settings, is shown in FIG. 10.

The analyses described herein overcome many disadvantages seem with traditional methods, e.g. time- and material-consuming procedures and risk of loss or contamination during sample preparation. The simplification of the sample purification step, by using GPC prior to HPLC analysis, reduces the risk of analyte loss or contamination. Automation of injection and, for multiple analytes, switching of detector settings, allows rapid and convenient analysis. With the fully automated analytical in-line method, 20 different PAHs (including the 13 FEDIOL PAHs) can be analyzed within 100 minutes, as shown above. The in-line GPC-HPLC technique drastically reduces analysis time and volume of solvents consumed and can be applied as a routine analysis to run on a continuous basis.

### EXAMPLES

Materials and Methods

GPC was carried out on a Waters Styragel®-HR 0.5 GPC column (4.3 mm I.D.×300 mm; 5μ particle size), using the HPLC-pump Model 515, Autosampler Model 717, Differential Refractometer Model 410, Diode Array detector Model 996 (or other UV detector) and the Fluorescence detector. The fluorescence detector is typically connected to the outlet of the RI-detector. For “fingerprint” fuel oil analysis, only the photodiode array detector was used.

GPC/HPLC (quantitative) analyses employed an HPLC pump Model 515, an Autosampler Model 717 with a 250 μl sample loop, a Differential Refractometer Model 410, a Photodiode Array detector Model 996, a Scanning Fluorescence detector Model 474, a Gradient pump Model 600, a Rheodyne 6-position fluid processor, and a column oven. A Waters Millennium Chromatography manager was used for data acquisition, processing and management of chromatographic information. The RP-HPLC column employed was a BESTER Analytical PAK (ODS) column 5 μm, 250×3.0 mm, with a Phenomenex® Security Guard C18 (ODS) precolumn 4×3.0 mm.

### Example 1

### Rapid “fingerprint” Analysis of B[a]p in Coconut Oil by GPC

A sample (about 8-12 drops) of crude coconut oil was dissolved in 7 ml of 5:2 THF/acetonitrile, and the solution was filtered through a 0.20 μm syringe filter and injected onto a Waters Styragel®-HR 0.5 GPC column (4.3 mm I.D.×300 mm; 5μ particle size). The sample was eluted with THF at a flow rate of 0.30 ml/min, and the fraction eluting at about 9.7 minutes was detected by fluorescence. Fluorescence detection was set at 378 nm excitation/403 nm emission, specific for benzo[a]pyrene. Total injected amount of sample was concurrently determined by refractive index detection.

A typical chromatogram is shown in FIG. 1. A calibration curve of fluorescence intensity vs. ppb B[a]p, prepared by running similar analyses of seven prepared standards, using Cargill Instrument Performance Monitoring software, is shown in FIG. 2.

The fluorescence of several other PAH at 378 nm/403 nm was examined to determine possible interference with B[a]P detection. See Table 6 below, which shows peak height relative to quantity of compound for a series of PAH (injected individually), with detection at 378/403 nm (optimized for detection of BaP). As can be seen, anthracene is the only compound of those shown giving a potentially significant amount of interference at these wavelengths. (Although benzo[k]fluoranthene absorbs more strongly at this wavelength than anthracene, it is generally present in negligible amounts compared to anthracene).

TABLE 6

| | Retn time | Amt injected | Peak height | Ratio |
|---|---|---|---|---|
| | (min.) | (ng) | 378 nm/403 nm | Peak height/Amt |
| Light PAH: | | | | |
| Fluorene | 9.37 | 73 | — | — |
| Acenaphthene | 9.35 | 71 | — | — |
| Phenanthrene | 9.31 | 915 | — | — |
| Anthracene | 9.48 | 41 | 21350 | 521 |
| Fluoranthene | 9.51 | 210 | 1850 | 8.8 |
| Pyrene | 9.93 | 282 | 241 | 0.9 |
| Benzo[a]anthracene | 9.29 | 75 | 1280 | 17 |
| Chrysene | 9.58 | 97 | — | — |
| Heavy PAH: | | | — | — |
| Benzo[b]fluoranthene | 9.32 | 30 | 1254 | 42 |
| Benzo[k]fluoranthene | 9.23 | 11 | 7120 | 647 |
| Benzo[a]pyrene | 9.88 | 31 | 94126 | 3036 |
| Benzo[ghi]perylene | 9.89 | 20 | 1870 | 94 |
| Dibenzo[ah]anthracene | 9.75 | 2.8 | — | — |
| Benzo[e]pyrene | 9.61 | 16 | — | — |
| Perylene | 9.78 | 210 | — | — |
| Coronene | 9.89 | 4 | — | — |

### Example 2

### Rapid Analysis of Fuel Oil in Palm Oil by GPC

A one gram sample of crude palm oil was mixed with 6 ml acetonitrile, and the mixture was agitated for several seconds and allowed to settle. The mixture was filtered through a 0.45 μm syringe filter into a sampler vial, and the acetonitrile (upper) phase was separated and injected onto the GPC column, as described for Example 1. The sample was eluted with THF at a flow rate of 0.30 ml/min, and the fraction eluting at about 12.2 minutes was detected by UV absorption at 226 nm.

FIG. 4 shows an overlay of GPC chromatograms of seven samples spiked with varying amounts of pure diesel oil and analyzed in this manner. A calibration curve of absorption vs. ppm diesel oil, prepared using Cargill Instrument Performance Monitoring software, is shown in FIG. 5.

### Example 3

### Quantitative Analysis of Benzo[a]pyrene in Edible Oil by GPC-HPLC

A solution of PAH in THF was obtained via GPC separation from coconut oil matrix components, as described above (Example 1). This fraction (approx. 0.3 mL) was transferred in-line to the HPLC system (Bester Analytical PAK ODS column (5 μm particle size, 250×3.0 mm) with a Phenomenex® Security Guard C18 (ODS) precolumn (4×3.0 mm)) via an injection switching interface consisting of a Rheodyne automated six-position fluid processor, a mixing tee, and an injection loop having a volume of 2 mL. During normal operation, as shown schematically in FIG. 6A, HPLC solvent (ACN/water) is pumped through the mixing tee and injection loop. During injection, as shown schematically in FIG. 6B, the PAH fraction in THF is mixed with the HPLC solvent in the mixing tee. Output of the solvent streams during injection is to the RI detector, which operates at atmospheric pressure. Accordingly, the pressure on the GPC column remains substantially constant.

After approximately one minute, the interface was switched back to operation mode (FIG. 6A), and the sample was eluted using the gradient program below:

TABLE 7

| | | Solvent A: | |
|---|---|---|---|
| | | Acetonitrile | Solvent B: |
| Time (min) | Flow (ml/min) | (%) | 5:95 ACN:Water (%) |
| 0.00 | 0.50 | 0 | 100 |
| 9.50 | 0.50 | 0 | 100 |
| 11.40 | 0.30 | 0 | 100 |
| 13.10 | 0.30 | 0 | 100 |
| 14.00 | 0.60 | 0 | 100 |
| 30.00 | 0.60 | 100 | 0 |
| 47.00 | 0.60 | 100 | 0 |
| 47.50 | 0.60 | 0 | 100 |
| 54.00 | 0.60 | 0 | 100 |
| 55.00 | 0.50 | 0 | 100 |

Under these conditions, benzo[a]pyrene eluted at about 51.3 minutes, and was detected by fluorescence at 378 nm/403 nm. A calibration curve was prepared from several standards of refined coconut oil spiked with benzo[a]pyrene at levels of 1-80 ppb.

### Example 4

### Detection of Multiple PAH by GPC-HPLC Using Programmed Detection

Calibration samples were prepared by spiking a standard mix containing 16 PAH (Restek; product M-610-QC in acetonitrile) into blank oil. The standard contained acenaphthene, acenaphthylene, anthracene, benzanthracene, benzo[a]pyrene, benzo[b]fluoranthene, benzoperylene, benzo[k]fluoranthene, chrysene, dibenzanthracene, fluoranthene, fluorene, indenopyrene, naphthalene, phenanthrene and pyrene. Benzo[a]fluoranthene, benzo[e]pyrene, perylene and coronene were obtained from Alltech Chemie. Standards were prepared having concentrations of B[a]p in the range of 0.2 to 4.2 ppb (μg/kg). These samples were used to determine retention time and detector response for each component to be analyzed.

Sample was accurately weighed and dissolved in 6 ml of 5:2 THF/acetonitrile. The solution was filtered through a 0.45 μm syringe filter into a sampler vial and used for injection. Preferred amounts of sample and injection volume for various types of samples are given below:

| Sample | Sample amt. | Injection volume |
|---|---|---|
| Crude oil | 0.10 g | 50 μl |
| Deodorized oil | 0.20 g | 100 μl |
| Distillate | 0.05 g | 20 μl |
| Standards | 0.20 g | 100 μl |

The apparatus used was as described in Materials and Methods, above. The GPC and HPLC columns were maintained at 40° C. throughout the procedure.

A volume of 100 μl of sample was injected onto the GPC column and eluted with THF at a flow rate of 0.30 ml/min. After about 11.0 minutes, the interface was switched to injection mode (FIG. 6B) for one minute. The sample was then transferred to the HPLC column (by switching back to operation mode; FIG. 6A), and the HPLC gradient, shown in Table 8 below, was initiated.

The fluorescence detector is programmed to switch to appropriate detection wavelengths for each component (PAH) at the predetermined retention time of that component. One such program is shown in Table 9 below. (Because acenaphthylene is not detectable by fluorescence, it may be detected by incorporating a UV detector.) Compounds were identified by comparing retention time with the previously injected standard and quantified by peak area measurement.

TABLE 8
Solvent Gradient

| Time | Flow, ml/min | Acetonitrile, % | 95:5 Water/Acetonitrile, % |
|---|---|---|---|
| 0 | 0.50 | 0 | 100 |
| 13.0 | 0.50 | 0 | 100 |
| 65.0 | 0.50 | 100 | 0 |
| 83.0 | 0.50 | 100 | 0 |
| 84.0 | 0.70 | 0 | 100 |
| 90.0 | 0.70 | 0 | 100 |
| 91.0 | 0.50 | 0 | 100 |
| 100.0 | 0.50 | 0 | 100 |

TABLE 9
Fluorescence Detection Timed Wavelength Program

| | Time, | Excitation, | Emission, | Attenua- | |
|---|---|---|---|---|---|
| Analyte | min. | nm | nm | tion | Gain |
| Start | 0 | 378 | 403 | 256 | 1 |
| Naphthalene | 57.0 | 280 | 370 | 8 | 100 |
| Acenaphthene | 62.3 | 302 | 330 | 32 | 10 |
| Fluorene | | 302 | 330 | | |
| Phenanthrene | 65.0 | 244 | 375 | | |
| Anthracene | | 244 | 375 | | |
| Fluoranthene | 67.9 | 280 | 460 | | |
| Pyrene | 69.2 | 330 | 388 | 128 | 10 |
| Benzanthracene | 73.0 | 280 | 420 | 32 | 10 |
| Chrysene | 75.0 | 261 | 400 | | |
| Benzo[a]fluoranthene | | 261 | 400 | | |
| Benzo[e]pyrene | 76.6 | 324 | 392 | | |
| Benzo[b[fluoranthene | | 324 | 392 | | |
| Perylene | 78.3 | 430 | 465 | | |
| Benzo[k]fluoranthene | 79.2 | 378 | 403 | | |
| Benzo[a]pyrene | | 378 | 403 | | |
| Dibenzanthracene | 82.9 | 290 | 440 | 8 | 100 |
| Benzoperylene | | 290 | 440 | | |
| Indeno-pyrene | 85.3 | 296 | 500 | 2 | 10 |
| Anthanthrene | 88.0 | 298 | 438 | | |
| Coronene | | 298 | 438 | | |

### Comparative Example

### Official Protocol for Determination of BAP in Edible Oils

The following procedure is taken from the “Code of Practice on Polycyclic Aromatic Hydrocarbons” of the French Society for Fat Science (FEDIOL).

Principle: A suitable amount of oil or fat in light petroleum is added to the top of a 22 g column of activity 4 alumina (capable or retaining about 600/mg oil or fat) and benzo[a]pyrene is eluted from the column with 75 ml light petroleum. Final analysis is by reversed phase high performance liquid chromatography using a fluorimetric detector.

Reagents

Light petroleum or hexane, analytical grade, redistilled over KOH pellets (4 g/l).
Acetonitrile and THF, LiChrosolv® (Merck).
Sodium sulfate, analytical grade, granular, anhydrous.
Alumina-activity 4 grade: Neutral aluminum oxide, activity super I supplied by Woelm, Eschwege, G.F.R., is deactivated by the addition of 10 ml water to 90 g alumina. Equilibrate for 24 h before use and store in a closed vessel at ambient temperature.

Benzo[a]pyrene, supplied by the Bureau Communautaire des References (BCR), Brussels, Belgium. Make a standard solution of about 0.1 μg/ml in tetrahydrofuran.

Apparatus

Glass chromatography columns: 30 cm×20 cm ID fitted with sintered discs and PTFE tops.

High pressure liquid chromatograph fitted with a fluorimetric detector.

Clean-Up

Prepare a 20% w/v solution of the oil sample in light petroleum.
Fill the chromatography column to the half with light petroleum. Rapidly weigh 22 g alumina, activity 4 grade, into a small beaker and transfer the alumina immediately to the column. Assist the alumina to settle by gently tapping the column.
Add anhydrous sodium sulfate on the top of the column in a layer of about 3 cm.
Open the top and run the level of the light petroleum just into the top of the salt layer.
Place a 10 ml measuring flask under the column.
Pipette 2.0 ml of the oil or fat solution onto the column with a minimum of rinsing, allowing the solvent layer to run into the sodium sulfate layer between additions.
Elute the column with light petroleum and discard the first 10 ml eluate.
Collect further 75 ml eluate in a 100 ml round bottom flask.
Concentrate the eluate with a Rotavapor to about 0.5-1 ml and transfer the solution into a crimp top minivial (volume 2 ml).
Continue evaporation in a water bath at 35° C. under a stream of nitrogen to dryness.
Dissolve the residue in 25 μl tetrahydrofuran. Stopper the minivial with a Teflon® layered septum and an aluminum cap and reserve for HPLC analysis with a fluorimetric detector.
   HPLC Analysis

The HPLC equipment consists of a liquid pump (e.g. Waters model 510), an injector (e.g. Waters U6K); a reversed phase column; a fluorimetric detector (e.g. Perkin Elmer 204A) and an integrator (e.g. HP-3390A). HPLC conditions are:
Eluent: Acetonitrile, flow 1 ml/min.
Column: Reversed phase HPLC column, Vydoc TP-RP, length 25 cm, I.D. 4.6 mm, particle size 10 μm (ex Chrompack)
Detector: Excitation wavelength 381 nm, Emission wavelength 403 nm.
   Inject 15 μl of the standard solution of benzo[a]pyrene.
   Inject 15 μl of the sample.
      Calculation

When the amount of sample is 400 mg, the final volume of the benzo[a]pyrene fraction is 25 μl, equal injection volumes (15 μl) of sample and standard are used, and:
h1=peak height in the sample
h2=peak height in the standard
c=amount benzo[a]pyrene (ng) in 1511 standard
p=benzo[a]pyrene level in μg/kg in the sample

$$p = \frac{h1 \times c25 \times 2.5}{h2 \times 15}$$

1. A method for determining the level of at least one polycyclic aromatic hydrocarbon (PAH) in a sample selected from edible oils, edible fats, and components thereof, the method comprising:
(a) providing said sample in a first solvent in which each said PAH is soluble;
(b) applying said sample to a gel permeation chromatography (GPC) column;
(c) eluting said sample with a GPC eluting solvent, effective to provide a fraction containing said PAH which is substantially free of triglyceride and free fatty acid components of said sample;
(d) injecting said fraction, without isolation, into a GPC/HPLC interface, wherein a solvent in which each said PAH has low solubility is added to said fraction;
(e) transferring said fraction, without isolation, onto a reverse-phase high performance liquid chromatography (HPLC) column,
(f) initially eluting said fraction with a solvent in which each said PAH has low solubility,
(g) separately eluting each said PAH to be detected from said HPLC column with an HPLC eluting solvent,
(h) detecting said at least one PAH; and
(i) determining the level of said at least one PAH in said sample.

2. The method of claim 1, wherein said at least one PAH is selected from naphthalene, methyl naphthalene, fluorene, acenaphthene, acenaphthylene, phenanthrene, anthracene, fluoranthene, pyrene, benzo[a]anthracene, chrysene, benzo[a]fluoranthene, benzo[b]fluoranthene, benzo[k]fluoranthene, benzo[a]pyrene, benzo[ghi]perylene, dibenzo[ah]anthracene, indeno[123-cd]pyrene, benzo[e]pyrene, perylene, benzoperylene, anthanthrene, coronene, and combinations thereof.

3. The method of claim 2, wherein said at least one PAH includes benzo[a]pyrene.

4. The method of claim 1, wherein said detecting employs a fluorescence detector.

5. The method of claim 4, wherein said detecting employs wavelength(s) characteristic of each said PAH to be detected.

6. The method of claim 1, wherein said first solvent comprises tetrahydrofuran (THF).

7. The method of claim 6, wherein said first solvent is a mixture of THF and acetonitrile in a ratio of 70:30 or greater.

8. The method of claim 1, wherein the GPC eluting solvent is THF.

9. The method of claim 1, wherein said solvent in which each said PAH has low solubility comprises 95 to 100% water and the remainder, if any, acetonitrile.

10. The method of claim 1, wherein said HPLC eluting solvent is a solvent gradient comprising varying ratios of water and acetonitrile.

11. The method of claim 3, wherein the level of said benzo[a]pyrene in said sample is less than 1 ppb.

12. The method of claim 3, wherein the level of said benzo[a]pyrene in said sample is less than 0.1 ppb.

13. The method of claim 1, wherein the recovery of said PAH from said sample is at least 99%.

14. The method of claim 1, wherein said injecting step (d) and transferring step (e) are initiated automatically by software control of a valve switching mechanism.

15. The method of claim 4, wherein said detecting comprises switching said fluorescence detector to said wavelengths for each said PAH, at a predetermined HPLC retention time for said PAH, in an automated manner.

16. The method of claim 1, wherein said at least one PAH includes at least 10 different PAH.

17. The method of claim 1, wherein said at least one PAH includes at least 20 different PAH.

18. A method for determining the level of benzo[a]pyrene in a sample selected from edible oils, edible fats and components thereof, the method comprising:
providing said sample in a solvent mixture comprising THF, acetonitrile, or a mixture thereof;
applying said sample to a gel permeation chromatography (GPC) column;
eluting said sample with THF, effective to provide an aromatic hydrocarbon containing fraction which is substantially free of triglyceride and free fatty acid components of said sample; and
measuring the fluorescence absorption of said fraction at wavelengths characteristic of benzo[a]pyrene.

19. The method of claim 18, wherein the level of benzo[a]pyrene in said sample is less than 10 ppb.

20. The method of claim 18, wherein the level of benzo[a]pyrene in said sample is less than 5 ppb.

21. The method of claim 18, wherein the recovery of benzo[a]pyrene from said sample is at least 99%.

22. A method for determining the level of aromatic hydrocarbons in a sample selected from edible oils, edible fats and components thereof, the method comprising:
providing said sample in a first solvent comprising THF, acetonitrile, or a mixture thereof;
applying said sample to a gel permeation chromatography (GPC) column;
eluting said sample with THF, effective to provide a aromatic hydrocarbon-containing fraction which is substantially free of triglyceride and free fatty acid components of said sample; and
detecting the level of said aromatic hydrocarbons in the fraction by UV absorption.

23. The method of claim 22, further comprising the step of estimating the level of fuel oil contamination in said sample, from a separately determined correlation between fuel oil level and UV absorption intensity of the fraction.

24. The method of claim 22, wherein the first solvent is acetonitrile, and the method further comprises extracting said sample with acetonitrile.

25. The method of claim 22, wherein said aromatic hydrocarbon fraction comprises one or more components selected from benzene, toluene, ethylbenzene, xylenes, naphthalene, methyl naphthalenes, phenanthrene, and acenaphthene.

26. The method of claim 22, wherein the level of said aromatic hydrocarbons in the fraction is less than 5 ppm.
